(19) United States
(12) Patent Application Publication
(10) Pub. No.: US 2002/0187181 A1
(43) Pub. Date: Dec. 12, 2002

(54) System for delivering cosmetics and pharmaceuticals

(75) Inventors: Kristin J. Godbey, Vadnais Heights, MN (US); Steven S. Kantner, St. Paul, MN (US); Matthew T. Scholz, Woodbury, MN (US)

Correspondence Address: Attentiion: Christopher D. Gram, Office of Intellectual Property Counsel, 3M Innovative Properties Company, P.O.Box 33427, St. Paul, MN, 55133-3427, US

(73) Assignee: 3M Innovative Properties Company

(21) Appl. No.: 09/854,824

(22) Filed: May 14, 2001

(51) Int. Cl. A61K 9/70; B32B 27/12

(52) U.S. Cl. 424/443; 442/123

(57) ABSTRACT

A novel device for the delivery of one or more active agents to a subject is disclosed. The device includes a water-soluble or water-dispersible polymeric carrier, an adhesive, one or more active agents and a support layer. Methods of manufacturing and use of said device also are disclosed.

### BACKGROUND OF THE INVENTION

It may often be desirable to treat a specific body surface such as skin, nails, hair or teeth with a pharmaceutical, cosmetic or decorative agent. In order to effect such treatments, a topical application of a lotion, cream, ointment, foam, powder, plaster, emulsion, bandage or adhesive patch containing one or more pharmaceutical, cosmetic or decorative agents can be applied to the body surface for which such treatment is desired.

Lotions, creams, ointments, foams, emulsions and powders may be undesirable compositions for delivery of active agents because of the ease with which they can be removed from a body surface such as the skin before the full benefit of the intended treatment is delivered. Such compositions are subject to physical removal from skin, for example, by contact with clothing or another part of the body. Such removal not only interferes with the intended treatment, but it also creates an undesirable mess on the clothing or body part that removes the composition. Also, for applications in which dosing is important, reliably dispensing a properly measured dose of the treatment is often difficult by its very nature, and is made even more difficult when the composition delivering the treatment may be removed before the treatment is complete. Such compositions also may be undesirable because they may leave the treated body surface feeling greasy, wet, sticky or slippery.

Bandages and adhesive patches have been used to deliver active agents to body surfaces in a manner that reduces premature removal of the active agent, allows more reliable dosing, and reduces mess. However, such treatment devices are often bulky and therefore may be uncomfortable for the user. Also, removal of the bandage or adhesive patch from the body surface after treatment is often uncomfortable or even painful.

Devices for delivery of pharmaceutical agents to mucosal surfaces may be water-soluble and, therefore, may dissolve after delivery of the pharmaceutical agents. For example, a film including a monolayer of a water-soluble polymer, active agent and, optionally, one or more additional components are mucoadhesive and may be used for rapid delivery of pharmaceutical or cosmetic agents to the mucosal lining of an oral cavity. Such films are designed for rapid dissolution in the oral cavity, thereby minimizing any prolonged discomfort for the user. Such devices are generally unsuited for use on dry body surfaces because they rely on the interaction between the film and moisture of the oral cavity provided by saliva and the mucosal lining in order to make the devices adhesive. Also, such devices are unsuited for any prolonged treatment since they are specifically designed for generally rapid solvation in the oral cavity.

Other water-soluble delivery devices are known that may be suited to certain degrees for topical delivery of pharmaceutical or cosmetic agents. For example, films including polyoxazoline polymer compositions and an adhesive layer may be used for the delivery of certain antimicrobial agents. However, tackifier from the adhesive layer may diffuse into the film layer during prolonged storage of such films, thereby transferring some of the adhesive character of the device from the adhesive layer to the film layer. Thus, some desired adhesion is lost from the adhesive layer and the film layer exhibits increased tack, thereby making the device more difficult to handle. A medicament may be delivered to a body surface through a film prepared from a suspension of a medicament, film-forming polymer and, optionally, a release agent or filler. However, such films generally do not adhere well to a dry body surface and, in the absence of a wetting step, active agents are not easily delivered to the dry body surface. Bathing preparations may be simultaneously delivered to a body surface and dissolved into bath water using a patch including a water-soluble adhesive sheet containing the bathing preparation and an optional water-soluble protective material. However, because such patches are dissolved in a bath, their effectiveness for direct delivery of a treatment to a defined, specific area is limited.

There exists an ongoing need for a single device capable of delivering a broad range of treatments to a variety of body surfaces.

### SUMMARY OF THE INVENTION

The present invention provides a single device for delivery of an active agent that may be designed to have properties advantageous for delivery of a large number and variety of active agents. Generally, the device of the present invention may be designed to be easy to apply and easy to remove. In some embodiments, the device may be designed to deliver a pre-measured unit dose of agent to a limited, specific area. Certain embodiments of the device may be applied to dry skin, hair or nails while other embodiments of the device may be applied to moistened surfaces such as teeth or mucous tissue. The device of the present invention may be designed variously for providing prolonged treatments or for dissolving or dispersing rapidly in water.

The present invention provides a device for delivering at least one active agent to a subject comprising: a substantially water-soluble or substantially water-dispersible carrier comprising at least one polymer and at least one plasticizer, and having a first surface and a second surface; a substantially water-soluble or substantially water-dispersible adhesive disposed on at least a portion of the first surface of the carrier, and having a carrier surface in contact with first surface of the carrier and an application surface generally opposed to the carrier surface; and at least one support layer releasably adhered to the application surface of the adhesive, the second surface of the carrier, or both. In another aspect, the device also includes at least one active agent in association with the carrier, the adhesive, or both.

The present invention also provides a method of delivering at least one active agent to a subject, the method comprising providing a delivery device comprising i) a substantially water-soluble or substantially water-dispersible carrier comprising at least one polymer and at least one plasticizer, and having a first surface and a second surface, ii) a substantially water-soluble or substantially water-dispersible adhesive disposed on at least a portion of the first surface of the carrier, and having a carrier surface in contact with first surface of the carrier, and an application surface generally opposed to the carrier surface, iii) at least one active agent in association with the carrier, the adhesive, or both, and iv) at least one support layer releasably adhered to the application surface of the adhesive, the second surface of the carrier, or both; adhering the device to the subject; allowing the active agent to be delivered to the localized body surface; and removing the device.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### Definitions

For purposes of this invention, the following definitions shall have the meanings set forth.

“A” or “an” refers to one or more of the recited elements.

“Active agent” refers broadly to any agent providing any treatment to a user, whether or not the agent possesses biological activity. Thus, active agents include topical pharmaceutical agents such as, but not limited to, antimicrobial and antifungal agents, steroids and other anti-inflammatories; systemic pharmaceutical agents such as, but not limited to, hormones, vitamins or drugs; and cosmetic agents such as, but not limited to, colorants, bleaching agents or decorative treatments.

“Associating,” “in association with” or any variation thereof shall include any mode of incorporating an active agent into or depositing an active agent onto a carrier or an adhesive. Such modes shall include, without limitation, instances in which the active agent forms a suspension or an emulsion in the carrier or adhesive or is adsorbed to or absorbed by the carrier or adhesive. An active agent also may be in association with a carrier or adhesive as a coating applied to a surface of the carrier or adhesive.

“Cold-water soluble” or “cold-water dispersible” shall mean that the material so defined is dissolved or dispersed, as the case may be, in water or other aqueous solution at a temperature of less than about 40° C. in less than about two minutes when a 2.5 cm×2.5 cm sample is immersed in 500 ml of water or other aqueous solution in a beaker, with gentle stirring (for example, agitation with a magnetic stirrer producing a vortex to 75% of the fill line).

“Plasticizer” refers broadly to any material that increases the flexibility of a polymeric film or fabric.

“Treatment” refers broadly to any desired effect provided by an active agent to a user. Treatments shall include pharmaceutical treatments such as but not limited to, delivery of drugs, hormones, antimicrobial agents, and the like; and cosmetic treatments such as, but not limited to, delivery of hair or skin colorants and transfer of ornamental designs, masks, tattoos or appliques.

### DETAILED DESCRIPTION OF THE INVENTION

The present invention provides a novel device for delivery of one or more pharmaceutical, cosmetic or decorative active agents. The device of the present invention is suitable for use in a wide variety of treatments and may be designed to be easy to handle, easy to use, and to deliver a treatment to a limited, specific area. The device of the present invention also may be designed to deliver systemic treatments, as described more fully below.

The device includes a substantially water-soluble or substantially water-dispersible carrier for the delivery of one or more active agents. The carrier may be a film, fabric, tape, or any other form suitable for delivery of the active agent. The device also includes an adhesive disposed on at least a portion of one surface of the carrier. The active agent may include one or more pharmaceutical, cosmetic, decorative or other suitable type of agent. The active agent may be coated onto, dissolved into, suspended in, emulsified with, or otherwise applied to the carrier, the adhesive, or both. The device optionally includes one or more support layers releasably adhered to the carrier, the adhesive or both. Consequently, as used herein, “device” refers to the combination of a carrier, the adhesive and at least one active agent, either with or without a support layer. A support layer, if present, may provide support and structure to the device, thereby making the device easier to handle.

As will be described in more detail below, the device of the present invention may be placed on a localized body surface, thereby providing localized delivery of the active agent. The device may be configured so that the delivery of the active agent is immediate or delayed, prolonged or short-lived. Depending upon the particular application, water may be applied to the device so that the carrier quickly dissolves or disperses to a desired extent. If the carrier is completely dissolved, it may be washed away, leaving only the active agent in cases where the active agent is less water-soluble than the carrier. If the carrier or adhesive is incompletely dissolved, it may serve as a binder for the active agent providing, for example, adhesion and substantivity. When used in this way, the binder and active agent may be rubbed into the skin, for example, to provide the desired treatment.

The device of the present invention has broad utility. The device may be employed to deliver treatments such as, but not limited to, acne treatments, corn, wart or callus removers, hair conditioners, teeth whiteners, or other treatments of the skin, hair, nails or teeth. The device also may have decorative utility, for example, as temporary tattoos, masks or decorative appliques on the skin, toenails, fingernails or teeth, or by delivering hair color or skin color. The device also can serve to cover blemishes, scars, or disfigurations, thereby providing a smoothed surface over which traditional powdered or liquid make-ups can be applied. The device also may have particular utility in delivering active agents such as antimicrobial agents, antibiotics, growth factors and the like to topical wounds such as burns and abrasions as well as chronic wounds. The device also may have particular utility in accelerating wound debridement by supplying enzymes or other active agents that can accelerate removal of necrotic tissue from affected wounds. The device of the present invention may have other utilities as well. It may be useful, as nonlimiting examples, for masking or camouflaging skin blemishes, cushioning sores, hair removal, or applying sunscreen or insect repellant. The claimed devices may also be useful for delivery of topical or systemic pharmaceutically active agents. If desired, an active agent may be provided in a unit dose amount when the device is manufactured.

The device of the present invention allows one to deliver an active agent in a substantially dry state to a specific area. Adhesive tapes, bandages and known patches may be applied to substantially dry skin, but they also may be very noticeable once applied, uncomfortable to wear, and painful to remove. In contrast, the device of the present invention includes a carrier that may be designed to be thin, flexible, substantially transparent and substantially water-soluble or substantially water-dispersible. Thus, the device of the present invention may be designed to be substantially unnoticeable once applied, thin and flexible enough to avoid causing discomfort while worn, and may be removed easily and painlessly after use. Also, because the device dissolves or disperses upon removal with water, no waste that may contain residual active agent is generated by using the device of the present invention. Consequently, small children and pets will not unintentionally be exposed to such waste. The device also may be designed to provide a delivery vehicle that will not stick to or be absorbed by clothing. When delivering active agents in which dosing is an important consideration, the device may be designed to allow for delivery of pre-measured doses of the active agent. In contrast to other water-soluble films, the device of the present invention may be used for prolonged treatments, may be applied to dry body surfaces without wetting, and is easy to handle. The adhesive and the carrier may be selected to substantially limit chemical or mechanical skin irritation and allow moisture to escape from the skin, thereby preventing maceration. Such construction allows for long-term wear of the devices.

The Carrier

The material used to prepare the carrier of the present invention may be any of the known natural or synthetic water-soluble or water-dispersible film-forming polymers and oligomers. In certain embodiments, the carrier material is selected to be cold water-soluble. Suitable polymers and oligomers include, but are not limited to, vegetable natural polymers such as alginic acid and alginic acid derivatized polymers, arabinogalactan, cellulose derivatives including but not limited to hydroxyethyl and hydroxypropyl cellulose, starch and starch derivatives; microorganism-derived natural polymers such as polysaccharides, polymers derived from animals including gelatin, collagen, mucopolysaccharides and the like; polyoxyalkylenes; polymers and copolymers derived from ethenically unsaturated monomers including, but not limited to, vinylic monomers, acrylates and methacrylates, acrylamides and methacrylamides, and the like; polyethyleneimines; and mixtures including one or more of the foregoing. Polymers of polyvinyl alcohols, polyvinyl pyrrolidone, proteins such as gelatin and collagen and derivatives thereof, or carbohydrates such as arabinogalactan have been recognized as having particular utility.

Polymers of polyvinyl alcohols may be prepared from polyvinyl acetate and can be commercially obtained in a variety of molecular weights and hydrolysis levels. The hydrolysis level determines, in part, whether the polymer is cold water-soluble or warm water-soluble, with hydrolysis greater than about 87% resulting in more crystalline polymers, thereby requiring higher temperatures to dissolve the polymer. The speed at which the polymer dissolves is determined, in part, by the molecular weight of the polymer and the presence of additional additives such as plasticizers or crosslinkers. One additional advantage of using polymers of polyvinyl alcohols to prepare the carrier film is that the film may, as a result of its low oxygen permeability, provide protection to oxygen sensitive materials such as vitamin C and its derivatives. In addition, certain plasticized polyvinylalcohol resins are thermoplastic and may be melt extruded or cast into films.

Plasticizers can be used to reduce the brittleness of the carrier film, thereby making the film tougher, more conformable and generally improving its handling properties. Certain plasticizers can also provide a degree of adhesiveness to the carrier, if desired. Using water alone as the plasticizer yields a carrier that is prone to rapid loss of moisture and a concomitant change into a glassy or brittle material when exposed to ambient conditions. Hence suitable plasticizers generally include alcohols, mixtures of alcohols, and mixtures of water and alcohols. Suitable plasticizers for use in the present invention include, but are not limited to, polyhydric alcohols such as glycerin, polyglycerol, alkyl polyglycosides, diethylene glycol, triethylene glycol, polyethylene glycol, random copolymers of ethylene oxide and propylene oxide, ethylene oxide/propylene oxide block copolymers such as those available from BASF under the Pluronic tradename, propylene glycol, sorbitol, sorbitol esters, butanediol, and their alkoxylated derivatives; monohydric alcohols such as 3-methoxy-3-methyl-1-butanol, alkyl ether ethoxylates, alkyl ester ethoxylates, aryl ether ethoxylates, aryl ester ethoxylates, aralkyl ether ethoxylates or aralkyl ester ethoxylates; urea, pyrrolidone carboxylic acids, pyrrolidone carboxylate salts, triethanol amine, ethanol acetamide, water, certain active agents such as vitamin E (α-tocopherol) and many common emollients; or any mixture including one or more of the foregoing. Non-polar active agents may be suspended or emulsified in the carrier by including a nonionic surfactant having a hydrophilic-lipophilic balance (“HLB”) value of at least about 8 as part or all of the plasticizer. Nonionic surfactants having an HLB value of at least about 12 have been shown to have particular general utility. The HLB value indicates the extent to which a given surfactant will behave as an oil-soluble versus a water-soluble type of emulsifier as described in “The Chemistry and Manufacture of Cosmetics,” Volume I, Third Edition, Mitchell L. Schlossman, Editor, Allured Publishing Corp., Carol Stream, Ill., 2000. Representative non-ionic surfactants include, without limitation, C8 to C22 alkyl ether ethoxylates, C8 to C22 alkyl ester ethoxylates, sorbitol C8-C22 alkyl esters, sorbitol C8 to C22 alkyl ester ethoxylates, and mixtures including one or more of the foregoing.

The amount of plasticizer present in the carrier may vary depending upon, among other things, the polymer used to form the carrier and the particular active agent or agents that also may compose the carrier. Some carriers may be at least about 5% plasticizer, by weight, although some carriers may be at least about 3% or at least about 1% plasticizer, by weight. Some carriers may be as much as 30% plasticizer, by weight, although other carriers may be as much as about 40% or as much as about 50% plasticizer, by weight. Certain carriers may include plasticizers in the range of about 5% to about 30% by weight. Such carriers generally provide good flexibility without compromising strength.

The carrier film may be prepared by dissolving at least one polymer and at least one plasticizer in water or other appropriate solvent. The solution thus prepared may be cast into a film, then dried. Water-soluble materials such as vitamin C, hydroquinone, and salicylic acid may be dissolved directly into the polymer solution. Water-insoluble materials such as vitamin E, benzoyl peroxide and silicone fluid may be emulsified into the polymer solution with an added surfactant. Alternatively, the active agent may be applied to the carrier film after it is cast and dried. In this case, the active agent is coated on the surface of the film. If certain characteristics are desired in the delivery device, additional additives may be combined with the polymer solution in order to impart the desired characteristics to the carrier film. For example, addition of low levels of silicone fluid or silicone copolyols provides carriers with a lubricious feel, addition of a biocide prevents mold or bacterial growth on the carrier during storage, and addition of particulate materials, such as the flattening agents used in the paint industry, provides a non-glossy matte finish to the dried carrier.

A water insoluble film-forming polymer also may be included in the carrier to improve its flexibility, strength or barrier properties as well as to adjust its solubility properties (e.g. dissolution time). One method of introducing this water-insoluble polymer is by adding an aqueous emulsion of the water-insoluble polymer to the solution of water-soluble polymer. If the water-soluble polymer is present in sufficient concentration, then the water-dispersibility of the resulting carrier is maintained.

Thermoplastic carriers may be embossed with heat, pressure, or both after drying to impart a texture or pattern to it. The carrier also may be cast and dried on a surface with a textured surface to provide, for example, a matte surface texture. Desired additives may therefore include, but are not limited to, surfactants, silicone oils, biocides, and particulate materials.

Fabrics useful as the carrier of the device may be constructed by any known technique for making woven, nonwoven, knitted, or other types of fabrics including open and closed cell foams. Nonwoven techniques include spun bonding, melt blowing, wet laying, hydroentangling (such as with cold water, relatively high salt concentration, or both), thermal bonding, or any combination of the foregoing. Polymeric fibers useful for the manufacture of the fabric are commercially available.

Alternatively, the films or fabrics can be melt processed with the appropriate polymer composition using known techniques. For example, certain plasticized polyvinyl alcohols may be melt processed. Heat-stable active agents may be added directly to the polymer melt. Alternatively, active agents may be coated onto or absorbed into a water-soluble or water-dispersible film or fiber using techniques such as those reported in U.S. Pat. No. 5,688,523, issued Nov. 18, 1997. Water-insoluble thermoplastic polymers may be included in the melt to alter the solubility, flexibility, strength, barrier, or other properties of the resulting carrier.

The particular form of the carrier and the materials used to prepare the carrier may be selected to provide the carrier with desired characteristics. For example, a thin, transparent film carrier may be desired for treatments requiring that the device be substantially unnoticeable in use. A woven or nonwoven fabric carrier may be desired for treatments in which high porosity is required. A film or higher basis weight nonwoven may be desirable for treatments in which a more substantial device is desired. Such treatments may include strips braided into hair for delivery of colorants, dyes or bleach or where printing is done on the carrier, such as for a mask.

The Adhesive

A wide variety of chemistries are known that provide water-soluble or water-dispersible adhesive compositions that are suitable for use in the present invention. Generally, such adhesives may include a lightly crosslinked or uncrosslinked polar polymer and a plasticizer in an amount sufficient to provide a degree of pressure sensitive tack. Suitable adhesives may or may not include water. Certain adhesives suitable for use in the present invention may be cold water-soluble. In one embodiment, the adhesive includes an uncrosslinked polar polymer and a compatible plasticizer in the absence of water. Such an adhesive provides good adhesion and rapid water-solubility without negatively affecting the film. In another embodiment, the adhesive includes a polymer of crosslinked polyvinyl pyrrolidone a glycol plasticizer and optionally water such as those reported in U.S. Pat. No. 4,931,282, issued Jun. 5, 1990, U.S. Pat. No. 5,225,473, issued Jul. 6, 1993, and U.S. Pat. No. 5,276,079, issued Jan. 4, 1994.

Polymers suitable for use in the adhesive include, but are not limited to, poly(ethylene oxide); natural and synthetic polysaccharides and their derivatives; and homopolymers and copolymers of ethylenically unsaturated hydrophilic monomers including ethylenic unsaturated carboxylic acids having 3 to 8 carbon atoms such as (meth)acrylic acid and salts thereof as well as polymers derived from polymerization and subsequent hydrolysis of unsaturated anhydrides such as maleic anhydride and itaconic anhydride; acrylamide, N-vinyl pyrrolidone, hydroxyethyl (meth)acrylate, acrylamidopropane sulfonic acid and salts thereof; methyl vinyl ether; ethyl vinyl ether; and polymers having ammonium functionality derived from reaction of amine containing monomers with alkylating agents or protic acids, for example N,N′-dimethylaminoethyl (meth)acrylate and its derivatives, and vinyl pyridine. In one embodiment of the device of the present invention, the polymer includes a homopolymer or copolymer of acrylic acid, wherein the acidic groups may be neutralized from 0.5 to 95%, as reported in U.S. Pat. No. 4,848,353, issued Jul. 18, 1989. Alkali hydroxides, such as sodium hydroxide or potassium hydroxide, may be used as a neutralizing agent for the acidic groups. In an alternative embodiment, the polymer includes a homopolymer or co-polymer of N-vinyl pyrrolidone. In another alternative embodiment, the polymer includes a cohesive, conformable, nonionic, hydrophilic synthetic polymer as reported in U.S. Pat. No. 4,273,135, issued Jun. 16, 1981. In yet another alternative embodiment, the polymer includes a cohesive, conformable, hydrophilic synthetic polymer containing at least 5 mole percent of monomer units containing the salt of a carboxylic acid as reported in U.S. Pat. No. 4,352,359, issued Oct. 5, 1982.

Polymers suitable for use in the adhesive may be an uncrosslinked polymer or mixture of polymers with an overall number average molecular weight between 10,000 and 100,000 daltons. Such polymers provide a good balance of cohesive strength and water-solubility.

The adhesive composition of the device of the present invention may include the polymer in a relative amount of from about 10 to about 60 weight percent of the adhesive composition. Certain embodiments of the present invention may include an adhesive composition including from about 20 to about 50 weight percent polymer. Adhesive compositions containing this level of hydrophilic polymeric matrix have a desirable balance of tack, softness, adhesiveness, and cohesive strength. The adhesive composition may have a substantially homogeneous appearance, i.e., the aqueous, liquid phase is retained in the polymeric matrix and essentially no phase separation can be observed with the eye.

The adhesive composition may further include a plasticizer that includes from about 10 to about 80 weight percent (relative to the total weight of the adhesive) polar organic compound and about 0 to 60 weight percent water. All of these weight percents are based on the total weight of the entire adhesive composition. Suitable compounds for use in the plasticizer include, but are not limited to, monohydric alcohols and polyhydric alcohols. Low molecular weight polyoxyethylenes (average molecular weight up to 600 daltons), glycerol, monomethoxypolyoxyethylene and propanediol are suitable because they give good adhesive performance.

The plasticizer also may include a compatible anionic, cationic, nonionic or amphoteric surfactant. The use of such surfactants improves the adhesion of the adhesive to oily surfaces by providing the adhesive lipophilic properties as reported in U.S. Pat. No. 6,121,508, issued Sep. 19, 2000. The compatibility between the adhesive and the oily surface is improved by incorporating the surfactants into the adhesive. The surfactant also may serve to make hydrophobic active ingredients more compatible with the adhesive.

The adhesive composition of the device of the present invention may contain the plasticizer in an amount up to about 80 weight percent and water in an amount up to about 60 weight percent. Certain embodiments may include plasticizer from about 10 to about 50 percent by weight and water up to about 10 percent by weight. Such adhesives generally have a good balance of pressure sensitive adhesive performance while maintaining good water solubility.

Active Agents

The device of the present invention may be designed to deliver one or more active 30 agents to a specific, limited body surface. For certain embodiments, a delivered active agent may remain localized at the site of delivery. For other embodiments, an active agent may enter the bloodstream in order to provide a systemic treatment.

A single device of the claimed invention may deliver any number of active agents. More than one active agent may be mixed together so long as each active agent is compatible with each of the other active agents being co-delivered by the same device. Alternatively, an active agent that reacts with a second active agent may be used, configured within the device to be separated from the second active agent by the carrier, the adhesive, or both and allowed to react only when the device is activated by moistening. This may be particularly useful for in situ mixing of, for example, baking soda and hydrogen peroxide for oral care.

One or more active agents may be delivered by the device of the present invention by being in association with the carrier film, the adhesive, or both as the device is applied to the desired body surface. The association between an active agent and the carrier film or adhesive may include, but is not limited to, as a coating, suspension, emulsion, or solution.

The device of the present invention may be useful for any of a large number and wide variety of treatments, some of which are described below. It should be understood that the description of possible treatments according to the present invention is intended to be exemplary in nature and is not intended to unduly limit the scope of the invention in any way. One skilled in the art will be able to design a device as disclosed herein with properties suitable for use in the described or any other treatments.

The device of the present invention may be used to deliver a broad assortment of active agents to the skin. The claimed device may be flexible and conformable, thereby providing comfortable treatment of the device to various skin contours. For skin treatments, it may be desirable that the device is able to adhere to dry skin, although application to wet or pre-moistened skin is also within the scope of the claimed invention. Adhesion of the device to dry skin allows the device to be used for various applications in which prolonged treatment may be desirable. For example, the device may be used to apply an active agent for an overnight skin treatment. In one embodiment, the device is applied to dry skin, provides prolonged treatment, and then is washed away easily and quickly after treatment is completed. Active agents that may be delivered to the skin in this manner include, but are not limited to, emollients, humectants, conditioners, moisturizers, vitamins, herbal extracts, antioxidants, steroids or other anti-inflammatory agents, vasodilators, exfoliants such as a-hydroxy acids or P-hydroxy acids, growth factors, enzymes, bleaching or coloring agents, antifungal or antimicrobial agents (including antibiotics and antiseptics such as povidone-iodine, chlorhexidine gluconate, triclosan, p-chloro-m-xyenol, fatty acid monoesters of glycerin and propylene glycol, benzoyl peroxide, hydrogen peroxide, silver and silver salts including, but not limited to, silver chloride, silver oxide and silver sulfadiazine, phenols, miconazole, clotrimazole, ketoconazole, econazole, undecylenic acid and the like), emulsifiers, artificial tanning agents, tanning accelerants, skin soothing agents, skin tightening agents, anti-wrinkle agents, skin repair agents, sebum inhibiting agents, sebum stimulators, protease inhibitors, anti-itch ingredients, agents for inhibiting hair growth, agents for accelerating hair growth, skin sensates, antiacne treatments, depilating agents, astringents, hair removers, or corn, callus or wart removers. Ornamental or decorative designs, colorants, tattoos or glitters also may be applied to skin in this manner. For example, the claimed device may be used to fashion water-removable masks for decorating at least a portion of the skin, including the face.

Alternatively, active agents may be delivered to the skin by at least partially activating the surface area of the device with water or other moisture. In this way, at least some of the adhesive, carrier, or both are dissolved or dispersed. For some treatments, it may be desirable to completely dissolve or disperse the adhesive and carrier, thereby providing immediate and complete delivery the active agent. Alternatively, for some treatments it may be desirable to dissolve or disperse only a portion of the carrier, adhesive, or both. The remaining carrier or adhesive can be rubbed into the skin along with the active agent, thereby serving as a binder providing some degree of substantivity and persistence for the active agent. Active agents that may be delivered to the skin in this manner include, but are not limited to, glitters, fragrances including aromatherapy agents, perfumes, sunscreen agents, insect repellants, deodorants and antiperspirants.

The device also may be used to provide various treatments to hair. Again, depending upon the particular application, treatments may be prolonged or immediate and the device of the present invention may be designed to provide the desired treatment. Because the device may be flexible and conformable, it may be used to deliver a wide variety of hair treatments. For example, the device may be braided into the hair in order to provide prolonged delivery of hair colorants or bleach. A device including a nonwoven fabric may provide better comfort for such an application. Braiding of one or more colored strips of the device into the hair, followed by activation with water can create a “tie-dyed” appearance. Other hair treatments that are possible with the device of the present invention include, but are not limited to, prolonged or immediate delivery of conditioners, moisturizers, humectants, antidandruff agents, vitamins, fragrances, perfumes, herbal extracts, hair colorants, bleaching agents, texturizers and decorative agents including glitters.

The device also may be used to provide treatment to fingernails or toenails. Decorative colorings or appliques may be delivered to nails with the claimed device in a manner similar to that described above for the similar treatments to skin and hair. Antifungal agents, antimicrobial agents, or other medicinal agents also may be delivered to the nails with the device.

The device also may be used to deliver treatments to moistened surfaces such as teeth or mucosal tissue. Because such treatments occur in a naturally moist environment, it may be desirable to design the device for such treatments so that it dissolves or disperses slowly. Examples of dental treatments include, but are not limited to, fluoridation, whitening, stain bleaching, stain removing, remineralizing to form fluorapatite, plaque removal, and tartar removal. Examples of suitable medicaments include, but are not limited to, hydrogen peroxide, carbamide peroxide, sodium fluoride, sodium monophosphate, pyrophosphate, chlorhexidine gluconate, polyphosphate, triclosan, enzymes, and combinations thereof. Other useful medicaments include, but are not limited to, anti-inflammatory agents, antimicrobial agents, emollients, flavorants, fresheners, antipruritics, and other agents for treating soft tissues.

The device also may have utility as a wound dressing, first aid bandage, or athletic tape wrap that may be removed gently and substantially without pain by soaking in water. These medical articles may include active agents such as, without limitation, antimicrobial agents, antibiotics or wound healing agents. These wound dressings may further include water-soluble absorbents.

The device of the present invention also may be used to deliver an active agent that provides a systemic treatment. Delivery of systemic active agents may be through the skin or mucosal tissue. For such a treatment, a device of the present invention carrying the systemically active agent is applied to a localized body surface. The application of the device may be for a prolonged period or, alternatively, the device and active agent may be rubbed into the skin or mucosal tissue to which the device is applied. The active agent is absorbed into the skin or mucosal tissue and passes into the bloodstream. The bloodstream carries the active agent throughout the body, thereby allowing the active agent to provide systemic treatment. Active agents that may be delivered in this manner to provide systemic treatments include, but are not limited to, hormones, vitamins, drugs such as those reported in U.S. Pat. No. 6,019,997, issued Feb. 1, 2000, and combinations thereof.

For all treatments, the active agents should be compatible with the carrier, adhesive and support layer. The active agents, adhesive and carrier should be selected so that each will remain stable during storage.

Support Layer

The device of the present invention may include one or more support layers releasably adhered to the carrier, the adhesive, or both. The support layer is typically removed from the carrier and the adhesive at about the time a treatment is initiated. Because the carrier and the adhesive of the device may be thin, flexible and conformable, a support layer may be used to provide structural support to the device, thereby making the device easier to handle. A support layer also may cover the adhesive until the user is prepared to apply the device to a localized body surface for treatment. In this way, a support layer may protect the adhesive layer from contact with surfaces other than the body surface selected for the desired treatment. This improves handling of the device prior to treatment and reduces mess. A second support layer may be adhered to the carrier to provide rigidity to the device after removal of the first support layer from the adhesive. This prevents the device from wrinkling or curling up on itself, allowing for smooth, easy placement onto skin. Once the device has been applied to the desired body surface, the second support layer may be removed. One method of producing such a supported device is reported in U.S. Pat. No. 6,169,224, issued Jan. 2, 2001.

The material used for the support layer is not limited. Suitable materials for use in the support layer include, but are not limited to, paper, foils, and polymeric films as well as multilayered laminates thereof. The support layer should be easily releasable from the carrier or adhesive so that the device may be applied to the body surface receiving treatment. The material for the support layer also may be coated with one or more materials designed to make the support layer easily releasable.

### EXAMPLES

The following examples have been selected merely to further illustrate features, advantages, and other details of the invention. It is to be expressly understood, however, that while the examples serve this purpose, the particular ingredients and amounts used as well as other conditions and details are not to be construed in a matter that would unduly limit the scope of this invention.

### Examples 1 through 6

### Incorporation of active agents into water-soluble films

Two types of polymer solutions were prepared. Unless otherwise indicated, all percentages are by weight. A 55% aqueous solution of 10,000 m.w. poly(vinyl pyrrolidone) (PVP, from Sigma-Aldrich Fine Chemicals, St. Louis, Mo.) was prepared by dissolving 55 g in 45 g deionized water. A 35% aqueous solution of 9,000-10,000 m.w. 80% hydrolyzed poly(vinyl alcohol) (9K PVA, from Sigma-Aldrich Fine Chemicals) was prepared by dissolving 35 g in 65 g deionized water.

The following solutions were prepared as active agents: (A) 10% salicylic acid in isopropanol, (B) 10% sodium ascorbyl phosphate (BASF Corporation, Mount Olive, N.J.) in water, and (C) a mixture of 5 g tocopherol acetate (Sigma-Aldrich Fine Chemicals) with 0.5 g of sorbitan laurate (Uniqema, New Castle, Del.).

Table 1 shows the amount of active agent added to 5 g of polymer solution to form each of six blends used to form water-soluble films. Each blend was coated onto polyester film, dried for 10 minutes at 65° C., and the visual appearance of the coating was assessed after cooling.

TABLE 1

| Example | Polymer | Active Agent | Film Characteristics |
|---|---|---|---|
| 1 | 55% PVP | 0.55 g A | Clear, flexible |
| 2 | 55% PVP | 0.55 g B | Clear, flexible |
| 3 | 55% PVP | 0.30 g C | clear, flexible, mottled surface |
| 4 | 35% PVA | 0.35 g A | clear, inflexible |
| 5 | 35% PVA | 0.35 g B | clear, inflexible |
| 6 | 35% PVA | 0.05 g C | clear, some dewets, inflexible |

These results show that water-soluble (A), alcohol-soluble (B), and water-insoluble (C) actives can be dissolved or dispersed in the carrier and that the carrier can be prepared conveniently from high solids solutions of two different polymers. Films made from PVP are flexible and easy to handle and process. Films made from PVA tend to be less flexible than films made from PVP.

### Examples 7 through 10

### Addition of Plasticizer to a Water-Soluble Film

Two polymer blends were used to form plasticized water-soluble films, as shown in Table 2. 35% 9K PVA was prepared as for Example 5, above. Also, 30% 13K PVA was prepared as follows. A 30% aqueous solution of 13,000 m.w. 87% hydrolyzed polyvinyl alcohol (13K PVA, Sigma-Aldrich Fine Chemicals ) was prepared by dissolving 30 g of 13K PVA in 70 g deionized water. 10 g of this solution was combined with 0.6 g of 10% salicylic acid in isopropanol.

A 25% aqueous solution of glycerin was prepared. 0.28 g of the glycerin solution was added to 10.7 g of 35% 9K PVA for Example 7 (final concentration, 2%), 0.70 g of the glycerin solution was added to 10.7 g of 35% 9K PVA for Example 8 (final concentration 5%). 0.24 g of the glycerin solution was added to 10.6 g of 30% 13K PVA for Example 9 (final concentration, 2%), 0.60 g of the glycerin solution was added to 30% 13K PVA for Example 10 (final concentration, 5%).

Coating and drying as described for Examples 1-6 gave clear films with Examples 7 and 9 (with 2% glycerin) still a bit brittle and Examples 8 and 10 (with 5% glycerin) providing softer, tougher, more flexible films. A drop or two of water allows one to disperse these films and rub them in without perceiving much tackiness during dry down.

TABLE 2

| Example | Polymer | Plasticizer | Film Characteristics |
|---|---|---|---|
| 7 | 35% 9 K PVA | 2% glycerin | Clear, moderately flexible |
| 8 | 35% 9 K PVA | 5% glycerin | Clear, soft and flexible |
| 9 | 30% 13 K PVA | 2% glycerin | Clear, moderately flexible |
| 10 | 30% 13 K PVA | 5% glycerin | Clear, soft and flexible |

These results demonstrate that the flexibility and strength of films made from PVA can be improved by adding low levels of plasticizer.

### Example 11

### Preparation of a water-soluble film with two actives

40 g of 9K PVA was dissolved in a mixture of 2 g glycerin and 58 g deionized water with heating and stirring. 10 g of this solution was charged with 1 g of arabinogalactan (Larex Company, White Bear Lake Township, Minn.) and 1 g of 10% salicylic acid in isopropanol yielding a hazy solution. Coating and drying as above gave a hazy somewhat brittle film.

### Examples 12 and 13

### Preparation of water-dispersible tapes with active in carrier and adhesive

20 g of the 9K PVA/glycerin/water solution prepared in Example 11 was charged with 1.6 g of 10% salicylic acid in isopropanol. This was coated to a wet thickness of 75 μm onto siliconized polyester liner and dried 7 minutes at 65° C. to provide the carrier for Example 12. Similarly, the carrier for Example 13 was prepared from a solution of 20 g of the 13K PVA/water solution prepared in Examples 9 and 10 mixed with 0.30 g glycerin and 1.2 g 10% salicylic acid in isopropanol. Adhesive containing active was prepared according to U.S. Pat. No. 5,276,079, issued Jan. 4, 1994 and U.S. Pat. No. 5,438,988, issued Aug. 8, 1995. 14 g of polyvinyl pyrrolidone powder that had been crosslinked via gamma irradiation was suspended in 26 g of 300 m.w. polyethylene glycol (PEG 300). 60 g of water was added while mixing with high shear with an Omni Macro Homogenizer (Omni International, Waterbury, Conn.). 20 g of the resulting 40% solution was mixed with 1.6 g of 10% salicylic acid in isopropanol and coated and dried as above. The carriers were then laminated to the adhesive to give tapes sandwiched between two polyester support layers. The laminates seemed to be quite stable with no migration of plasticizer between the two layers apparent.

### Examples 14 through 18

### Preparation of Water-Dispersible Tapes with Active Only in the Adhesive

The adhesive coating from Examples 12 and 13 was laminated to a strip of plasticized polyvinyl alcohol film (Solublon SA-17 from Mitsui Plastics, White Plains, N.Y.) for Example 14. Another tape (Example 15) was prepared with a low tack adhesive obtained from a solution of 20 g uncrosslinked polyvinyl pyrrolidone (PVP K30 from BASF, Mount Olive, N.Y.), 10 g deionized water, 8 g of 400 m.w. polyethylene glycol (PEG 400), and 2.8 g of 20% salicylic acid in isopropanol, coated and dried as above. Another tape (Example 16) was prepared with a skin temperature activated adhesive obtained from a solution of 10 g PVP K30, 10 g deionized water, 5 g PEG 400, 2 g Brij 56 (Uniqema, New Castle, Del.), and 3.4 g of 20% salicylic acid in isopropanol, coated and dried as above. Another tape (Example 17) also was prepared with an alternative high tack adhesive obtained from a solution of 20 g PVP K30, 10 g PEG 400, 10 g deionized water, and 3 g 20% salicylic acid in isopropanol, coated and dried as above. This adhesive was also laminated to a textured water-soluble plasticized polyvinyl alcohol film (Monosol E6030 from Chris Craft). The resulting tapes had good adhesion to skin and could be worn comfortably for several hours, then removed with water. Alternatively, after adhering to skin, the tape could be treated with a few drops of water and rubbed-in to give a flexible, durable film that may be removed by rinsing with more water.

The complete disclosures of the patents, patent documents and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A device for delivering at least one active agent to a localized body surface comprising:
a substantially water-soluble or substantially water-dispersible carrier comprising at least one polymer and at least one plasticizer, and having a first surface and a second surface;
a substantially water-soluble or substantially water-dispersible adhesive disposed on at least a portion of the first surface of the carrier, and having a carrier surface in contact with first surface of the carrier and an application surface generally opposed to the carrier surface; and
at least one support layer releasably adhered to the application surface of the adhesive, the second surface of the carrier, or both.

2. The device of claim 1 wherein the carrier comprises a polymeric film, a woven fabric, a knitted fabric, a nonwoven fabric, an open cell foam or a closed cell foam.

3. The device of claim 1 wherein the at least one polymer is made from one or more substantially water-soluble or substantially water-dispersible monomers.

4. The device of claim 1 wherein the at least one polymer comprises a polyvinyl alcohol, a polyvinyl pyrrolidone, a protein, a carbohydrate, an alginic acid, a polyethyleneimine, a polyoxyalkylene, a polyacrylate, a polymethacrylate, a polyacrylamide, a polymethacrylamide, a homopolymer of ethenically unsaturated monomers, or a copolymer comprising ethenically unsaturated monomers.

5. The device of claim 4 wherein the protein is collagen, gelatin, any derivative thereof, or any combination of any of the foregoing.

6. The device of claim 4 wherein the carbohydrate comprises arabinogalactan.

7. The device of claim 1 wherein the at least one plasticizer comprises a monohydric alcohol, a polyhydric alcohol, polyethylene glycol, a polyether, a surfactant, an amide, a lactam, an amine, an amine salt, a-tocopherol, or any mixture including any of any of the foregoing.

8. The device of claim 7 wherein the monohydric alcohol is 3-methoxy-3-methyl-1-butanol, alkyl ether ethoxylate, alkyl ester ethoxylate, aryl ether ethoxylate, aryl ester ethoxylate, aralkyl ether ethoxylate, or aralkyl ester ethoxylate.

9. The device of claim 7 wherein the polyhydric alcohol is glycerin, polyglycerol, alkyl polyglycoside, diethylene glycol, triethylene glycol, polyethylene glycol, a random copolymer of ethylene oxide and propylene oxide, a block copolymer of ethylene oxide and propylene oxide, propylene glycol, sorbitol, a sorbitol ester, butanediol, or an alkoxylated derivative of any of the foregoing.

10. The device of claim 1 wherein the adhesive comprises a pressure sensitive adhesive.

11. The device of claim 10 wherein the pressure sensitive adhesive comprises a mixture of:
i) at least one substantially water-soluble or substantially water-dispersible polymer, and
ii) at least one plasticizer in an amount sufficient to provide a desired degree of pressure sensitive tack.

12. The device of claim 11 wherein the at least one substantially water-soluble or substantially water-dispersible polymer is made from at least one substantially water-soluble or substantially water-dispersible monomer.

13. The device of claim 11 wherein the at least one substantially water-soluble or substantially water-dispersible polymer is poly(ethylene oxide), a polysaccharide, a polysaccharide derivative, a homopolymer of ethenically unsaturated monomers, or a copolymer comprising ethenically unsaturated monomers.

14. The device of claim 1 wherein the at least one support layer is paper, a polymeric film, a foil, or any combination of the foregoing.

15. A system for delivering at least one active agent to a localized body surface comprising:
a substantially water-soluble or substantially water-dispersible carrier comprising at least one polymer and at least one plasticizer, and having a first surface and a second surface;
a substantially water-soluble or substantially water-dispersible adhesive disposed on at least a portion of the first surface of the carrier, and having a carrier surface in contact with first surface of the carrier, and an application surface generally opposed to the carrier surface;
at least one active agent in association with the carrier, the adhesive, or both; and
at least one support layer releasably adhered to the application surface of the adhesive, the second surface of the carrier, or both.

16. The system of claim 15 wherein the at least one active agent is effective for treatment of skin, hair, fingernails, toenails, teeth, or mucosal tissue.

17. The system of claim 16 wherein the at least one active agent is a dye, a pigment, a bleaching agent, or a hair colorant.

18. The system of claim 16 wherein the at least one active agent is a breath freshener, hydrogen peroxide, carbamide peroxide, sodium fluoride, sodium monophosphate, pyrophosphate, chlorhexidine gluconate, polyphosphate, triclosan, a flavorant, a fluoridating agent, a teeth whitening agent, a dental stain remover, a plaque remover, or a tartar remover.

19. The system of claim 16 wherein the at least one active agent is a glitter, an ornamental design, a mask, an applique or a tattoo.

20. The system of claim 16 wherein the at least one active agent is a fragrance, a perfume, an emollient, a humectant, a conditioner, a moisturizer, a surfactant, an herbal extract, a skin colorant, a color cosmetic, an emulsifier, a skin soothing agent, a skin tightening agent, an artificial tanning agent, a tanning accelerant, an anti-wrinkle agent, an exfolient, a sebum inhibiting agent, a sebum stimulator, a protease inhibitor, an anti-itch ingredient, an agent for inhibiting hair growth, an agent for accelerating hair growth, a hair remover, a skin sensate, a depilating agent or an astringent.

21. The system of claim 16 wherein the at least one active agent is a sunscreen agent, an insect repellant, an antiperspirant or a deodorant.

22. The system of claim 16 wherein the at least one active agent is a drug, a vitamin, a hormone, an antioxidant, an anti-inflammatory agent, a steroid, an antipruritic agent, an antifungal agent, an antibiotic, an antimicrobial agent, an antidandruff agent, an antiacne agent, a skin repair agent, a callus remover, a wart remover or a corn remover.

23. The system of claim 15 wherein the at least one active agent is associated with the carrier.

24. The system of claim 23 wherein the at least one active agent forms a coating on at least one surface of the carrier.

25. The system of claim 23 wherein the at least one active agent is dissolved, suspended, or emulsified within the carrier.

26. The system of claim 15 wherein the at least one active agent is associated with the adhesive.

27. The system of claim 26 wherein the at least one active agent forms a coating on the application surface of the adhesive.

28. The system of claim 26 wherein the at least one active agent is dissolved, suspended, or emulsified within the adhesive.

29. The system of claim 15 wherein the carrier comprises a polymeric film, a woven fabric, a nonwoven fabric, an open cell foam or a closed cell foam.

30. The system of claim 15 wherein the at least one polymer is made from one or more substantially water-soluble or substantially water-dispersible monomers.

31. The system of claim 15 wherein the at least one polymer comprises a polyvinyl alcohol, a polyvinyl pyrrolidone, a protein, a carbohydrate, an alginic acid, a polyethyleneimine, a polyoxyalkylene, a polyacrylate, a polymethacrylate, a polyacrylamide, a polymethacrylamide, a homopolymer of ethenically unsaturated monomers, or a copolymer comprising ethenically unsaturated monomers.

32. The system of claim 15 wherein the at least one plasticizer comprises a monohydric alcohol, a polyhydric alcohol, polyethylene glycol, a polyether, a surfactant, an amide, a lactam, an amine, an amine salt, a-tocopherol, or any mixture including any of any of the foregoing.

33. The system of claim 32 wherein the monohydric alcohol is 3-methoxy-3-methyl-1-butanol, an alkyl ether ethoxylate, an alkyl ester ethoxylate, an aryl ether ethoxylate, an aryl ester ethoxylate, an aralkyl ether ethoxylate, or an aralkyl ester ethoxylate.

34. The system of claim 32 wherein the polyhydric alcohol is glycerin, a polyglycerol, an alkyl polyglycoside, diethylene glycol, triethylene glycol, a polyethylene glycol, a random copolymer of ethylene oxide and propylene oxide, a block copolymer of ethylene oxide and propylene oxide, propylene glycol, sorbitol, a sorbitol ester, butanediol, or an alkoxylated derivative of any of the foregoing.

35. The system of claim 15 wherein the adhesive comprises a pressure sensitive adhesive.

36. The system of claim 35 wherein the pressure sensitive adhesive comprises a mixture of:
i) at least one substantially water-soluble or substantially water-dispersible polymer, and
ii) at least one plasticizer in an amount sufficient to provide a desired degree of pressure sensitive tack.

37. The system of claim 36 wherein the at least one substantially water-soluble or substantially water-dispersible polymer is made from at least one substantially water-soluble or substantially water-dispersible monomer.

38. The device of claim 36 wherein the at least one substantially water-soluble or substantially water-dispersible polymer comprises poly(ethylene oxide), a polysaccharide, a polysaccharide derivative, a homopolymer of ethenically unsaturated monomers, or a copolymer comprising ethenically unsaturated monomers.

39. The system of claim 15 wherein the at least one support layer is paper, a polymeric film, a foil, or any combination of any of the foregoing.

40. A method of making a device for delivery of at least one active agent to a localized body surface, the method comprising:
providing a water-soluble or water-dispersible carrier having a first surface and a second surface;
dissolving at least one water-soluble or water-dispersible polymer in a solvent to form a solution;
selecting a plasticizer such that when the plasticizer is added to the solution and the solution is dried to form a pressure sensitive adhesive, the plasticizer will provide a desired degree of pressure sensitive tack;
adding the plasticizer to the solution is an amount sufficient to provide a desired degree of pressure sensitive tack to the pressure sensitive adhesive.
drying the solution to form the pressure sensitive adhesive;
adding at least one active agent to the carrier, the pressure sensitive adhesive, or both;
applying the pressure sensitive adhesive to at least a portion of the first surface of the carrier, thereby defining a carrier surface of the pressure sensitive adhesive in contact with the first surface of the carrier and an application surface of the pressure sensitive adhesive generally opposed to the carrier surface; and
removably adhering at least one support layer to the second surface of the carrier, the application surface of the pressure sensitive adhesive, or both.

41. The method of claim 40 wherein the carrier further comprises at least one active agent.

42. The method of claim 40 wherein adding the at least one active agent to the pressure sensitive adhesive comprises adding at least one active agent to the solution before drying.

43. The method of claim 40 wherein the at least one active agent is a coating on at least one surface of the carrier, the pressure sensitive adhesive, or both.

44. The method of claim 40 wherein the at least one active agent is effective for treatment of skin, hair, fingernails, toenails, teeth, or mucosal tissue.

45. A method of delivering at least one active agent to a localized body surface, the method comprising:
providing a delivery device comprising
i) a substantially water-soluble or substantially water-dispersible carrier comprising at least one polymer and at least one plasticizer, and having a first surface and a second surface,
ii) a substantially water-soluble or substantially water-dispersible adhesive disposed on at least a portion of the first surface of the carrier, and having a carrier surface in contact with first surface of the carrier, and an application surface generally opposed to the carrier surface,
iii) at least one active agent in association with the carrier, the adhesive, or both, and
iv) at least one support layer releasably adhered to the application surface of the adhesive, the second surface of the carrier, or both;
adhering the device to the subject;
allowing the active agent to be delivered to the localized body surface; and
removing the device.

46. The method of claim 45 further comprising rubbing the active agent into the localized body surface.

47. The method of claim 45 wherein adhering the device to the localized body surface comprises adhering the device to dry skin, dry hair, a dry fingernail or a dry toenail.

48. The method of claim 47 wherein allowing the active agent to be delivered to the localized body surface comprises leaving the device in contact with the localized body surface for at least about one hour before removing the device.

49. The method of claim 45 wherein the at least one active agent provides a systemic treatment.

50. The method of claim 45 wherein removing the device comprises dissolving or dispersing the device in an aqueous medium.
